SEQUENCE LISTING (27 sequences)

SEQ ID NO: 1 - DNA, 309 bp, Bos sp.
atgccgaccgtcattagcgcctctgtggccccacggacaggggctgagcccakgtccccagggcccatcgcccaggcagcccaggacaagggcaccgaggcagggggtggaaacccaagtggcatctactcagccatcatcagccgcaattttcccattattggggtgaaagagaagacattcgagcagcttcacaagaaatgtctggaaaagaaggttctttttgtggatcctgagttcccaccggacgagacctccctgttttacagccagaagttccccatccagttcgtctggaagagacctccg

SEQ ID NO: 2 - DNA, 1201 bp, Bos sp.
tatataaaaacggagaggacggagctgggggctaacctcttcaccatcggtttcgccatctacgaggtgtgccgtccctgacgagcttcagaccgcctctttagggaagaggctttcagggggcttcctgaggggttccccagtttccccaacatccagtgccccccaaaactactgatatcaggcccacttgagtcagaacctcttagaagtttgtaaccagtgggggaagaacccacctaggggaggtgggggtggggctggggctgtccaggtaggtacatggggcattagagcccctcattcgctctacgattgctagcatgccctttggcaagtttccatcccgattaagacctgaaatccacaaaaatctgagcaccttccttttctgtttctccccaaggttcccaaagaggtatagaagtggagcagccagcagtgtgtgcagcgttaccagggtcctgggtccgcctggggcacatcagggcacctcctgtggggtttgggggcaggactgtgataggagagggcctggcccaccttacctaactccagagcaggtcctcgggccattgcatggcctcctgacccccaccccacaacatgcccttccccttgcccaccagagaccattacackctcacatgcttgctcacactcacacacagaatcacacagacacattgtgaggctgatcgccttctttgggggtggaggaatcacctccctcagacccagtccacaacccccacccctgcctccctggggtctttccccagtccagaggtgttctggagctgagcagcagcaattctggtaagtgaccctgagtgggggatgccagacttgaccttcacttctggatcacagcagaaaaggaagaggatatggggtagggagctccagggatgttgagctattcgaggccttgggaattggaagctggtaggttgtgactatgaaatgagtgaccagggagttgggaaggaggacccctctggaggaggactgtgagcaggacagggtgtccctcccaaggggctgaggtaccttgggctcttctctcccttagatgcacggcaacaagcagcacctgcagaaggacttcttcctgtacaatgcctccaaggctaggagcagcccctacatcaacatgcgggaggtgtctgagcgcttccgcctgctacaccaatattaa

SEQ ID NO: 3 - DNA, 689 bp, Bos sp.
tttttgggaaaaacatcaaactcaatgttctcgaggtaaagcctaacagtacactccccccgcactttaaaatttgaggtggagggatgggattggatgggatgggaaaggaacataaagggaagctcagagctcagcaggatgtggtcatggggaacgcatagtgggggaaggctctaaggaagcctcagttatggctttatgctggaaaatctgtactttctaggggagatctgtgggaaggatgcttctcaggaaatcttccstgtctggcatgctgggactggccttttcttctacgttttagatatgtagggcttggctcttttgtctcattgtgcattctgatcttgggacttcctcttgcagtggctgcagctcaccatgtatgcctgaaccaagctggccacatcgaaggcatggaggatcactcaggatttcaatttcacccaacagagctagtcacttacctcaaaggacacagttgctgcacccccaggaagcctccaggcacctcatcagtctagttcctcctctactcacttccctcccagccttagggcatctgtccatttgtcaggcccagcctgacccttcagttaaggaatggggaagggagagctgtgttgtccctgtgccacatggagccaagtgcctctgtctgcttccgctagccacgactacattta

SEQ ID NO: 4 - DNA, 3064 bp, Bos sp.
tccaggaaaaaatgatggcttccggaccacagctcagtttttaagctggacgaccttctgatgggttttaaacttcgaattggatgtggacatttttctttctgatgacagaatcactggaacttcccctttgcagttgcttcctttccttaaaggtagctgaatcttgctgtctttaaaaaccttttctttccaaatttgcctgccatgccgaccgtcattagcgcctctgtggccccacggacaggggctgagcccatgtccccagggcccatcgcccaggcagcccaggacaagggcaccgaggcagggggtggaaacccaagtggcatctactcagccatcatcagccgcaattttcccattattggggtgaaagagaagacattcgagcagcttcacaagaaatgtctggaaaagaaggttctttttgtggatcctgagttcccaccggacgagacctccctgttttacagccagaagttccccatccagttcgtctggaagagacctccggaaatttgtgagaatccccgatttatcgttggtggagccaatagaactgacatctgccaaggagatctaggggactgctggtttcttgcagccatcgcttgcctgaccttgaacaagcgtctgcttttccgggtcataccccatgatcagagtttcaccgaaaactacgcggggatttttcacttccagttctggcgctatggagactgggtggacgtggttattgatgactgcctgccaacctacaacaatcaactggttttcaccaaatccaaccatcgcaatgagttctggagtgctctgctggagaaggcttatgctaagctccatggttcgtatgaagccctgaaaggtgggaacactacagaggccatggaggacttcacgggaggagtgacagagttttttgaaatcaaggatgctcccagagacatgtacaagatcatgaagaaagccatcgagaggggttccctcatgggctgctccattgatgatggcacaaacatgacctatggaacctctccttctgggctgaaaatgggggagttgattgagcggatggtgaggaatatggataactcgcggctcagggactcagacctcatccctgagggatgctcagatgacagaccaactcggatgattgttccagttcagtttgagacaagaatggcctgtgggctggtcaaaggccatgcctactcagtcactgggctggaggaggccctgtacaagggtgagaaagtgaagctggtgcggctgcggaacccctggggccaggtggagtggaatggctcctggagtgacagctggaaggactggagctatgtggacaaggacgagaaggcccgtttgcagcaccaggtcactgaggatggagagttctggatgtcctacgatgattttatctaccatttcacaaagctggagatctgcaacctcacagctgatgccctggagtccgacaagcttcagacttggacagtgtccgtgaatgagggccgctgggtgaggggctgctctgccggaggctgccgcaacttcccagacactttctggaccaacccacagtaccgtctgaagctcctagaggaggacgacgaccccgatgattccgaggtgatctgtagtttcctggtggctctgatgcagaagaaccggaggaaggaccggaagctgggggctaacctcttcaccatcggtttcgccatctacgaggttcccaaagagatgcacggcaacaagcagcacctgcagaaggacttcttcctgtacaatgcctccaaggctaggagcagaacctacatcaacatgcgggaggtgtctgagcgcttccgcctgcctcccagcgagtacgtcattgtgccctccacttacgagccccaccaggagggcgagttcatcctccgggtcttctcggaaaagaggaacctctctgaggaagttgagaatacaatctctgtggatcggccagtgaaaaagaaaaaaaacaagcccatcatctttgtttcagaccgagcaaacagcaacaaggagctgggtgtggaccaggaaacagaggagggaaaagacaacacaagccctgataagcaagcaaaatccccacagctagagcctggcaacaccgaccaggaaagtgaggaacagcggcaattccggaatattttcaggcagatagcaggcgatgacatggagatctgcgcagatgagctcaagaacgtccttaacagagttgtgaacaaacataaggacctgaagacacaaggcttcacgctggagtcctgccgtagcatgattgctctcatggacacagatggctctgggagactgaacctgcaagagtttcatcacctctggaagaagattaagacgtggcagaaaattttcaaacactatgacacagaccaatctggcaccatcaacagctacgagatgcgcaatgcagtcaaagatgcaggcttccacctcaacaaccagctctacgatatcattaccatgcgctatgcagacaagtacatgaatattgacttcgacagtttcatctgctgctttgtcaggctggagggcatgttcagagctttcaatgcatttgacaaggatggggacggtatcatcaaactcaatgttctcgagtggctgcagctcaccatgtatgcctgaaccaagctggccacatcgaaggcatggaggatcactcaggatttcaatttcacccaacagagctagtcacttacctcaaaggacacagttgctgcacccccaggaagcctccaggcacctcatcagtctagttcctcctctactcacttccctcccagccttagggcatctgtccatttgtcaggcccagcctgacccttcagttaaggaatggggaagggagagctgtgttgtccctgtgccacatggagccaagtgcctctgtctgcttccgctagccacaggccagtgagagctttgcttggagtctgatggcctcagctctgaagacaagtactctccttagatgcttgcagctgtttgcagaagcatttgccaatggcaaaaa

SEQ ID NO: 5 - DNA, 450 bp, Bos sp.
accgaggcagggggtggaaacccaagtggcatctactcagccatcatcagccgcaattttcccattattggggtgaaagagaagacattcgagcagcttcacaagaaatgtctggaaaagaaggttctttttgtggatcctgagttcccaccggacgagacctccctgttttacagccagaagttccccatccagttcgtctggaagagrcctccggaaatttgtgagaatccccgatttatcgttggtggagccaatagaactgacatctgccaaggagatctaggggactgctggtttcttgcagccatcgcttgcctgaccttgaacaagcgtctgcttttccgggtcataccccatgatcagagtttcaccgaaaactacgcggggatttttcacttccagttctggcgctatggagactgggtggacgtggttattgatgactgc

SEQ ID NO: 6 - DNA, 450 bp, Bos sp.
gagccaatagaactgacatctgccaaggagatctaggggactgctggtttcttgcagccatcgcttgcctgaccttgaacaagcgtctgcttttccgggtcataccccatgatcagagtttcaccgaaaactacgcggggatttttcacttccagttctggcgctatggagactgggtggacgtggttattgatgactgcctgccaacytacaacaatcaactggttttcaccaaatccaaccatcgcaatgagttctggagtgctctgctggagaaggcttatgctaagctccatggttcgtatgaagccctgaaaggtgggaacactacagaggccatggaggacttcacgggaggagtgacagagttttttgaaatcaaggatgctcccagagacatgtacaagatcatgaagaaagccatcgagaggggttccctcatgggctgct

SEQ ID NO: 7 - DNA, 450 bp, Bos sp.
ccgcctgcctcccagcgagtacgtcattgtgccctccacttacgagccccaccaggagggcgagttcatcctccgggtcttctcggaaaagaggaacctctctgaggaagttgagaatacaatctctgtggatcggccagtgaaaaagaaaaaaaacaagcccatcatctttgtttcagaccgagcaaacagcaacaaggagctgggtgtggaccaggaaacagaggagggaaaagacracacaagccctgataagcaagcaaaatccccacagctagagcctggcaacaccgaccaggaaagtgaggaacagcggcaattccggaatattttcaggcagatagcaggcgatgacatggagatctgcgcagatgagctcaagaacgtccttaacagagttgtgaacaaacataaggacctgaagacacaaggcttcacgctggagtcctg

SEQ ID NO: 8 - DNA, 450 bp, Bos sp.
aaaaaacaagcccatcatctttgtttcagaccgagcaaacagcaacaaggagctgggtgtggaccaggaaacagaggagggaaaagacaacacaagccctgataagcaagcaaaatccccacagctagagcctggcaacaccgaccaggaaagtgaggaacagcggcaattccggaatattttcaggcagatagcaggcgatgacatggagatctgcgcagatgagctcaagaaygtccttaacagagttgtgaacaaacataaggacctgaagacacaaggcttcacgctggagtcctgccgtagcatgattgctctcatggacacagatggctctgggagactgaacctgcaagagtttcatcacctctggaagaagattaagacgtggcagaaaattttcaaacactatgacacagaccaatctggcaccatcaacagctacgagat

SEQ ID NO: 9 - DNA, 450 bp, Bos sp.
agctgggtgtggaccaggaaacagaggagggaaaagacaacacaagccctgataagcaagcaaaatccccacagctagagcctggcaacaccgaccaggaaagtgaggaacagcggcaattccggaatattttcaggcagatagcaggcgatgacatggagatctgcgcagatgagctcaagaacgtccttaacagagttgtgaacaaacataaggacctgaagacacaaggcttcacrctggagtcctgccgtagcatgattgctctcatggacacagatggctctgggagactgaacctgcaagagtttcatcacctctggaagaagattaagacgtggcagaaaattttcaaacactatgacacagaccaatctggcaccatcaacagctacgagatgcgcaatgcagtcaaagatgcaggcttccacctcaacaaccagctctacg

SEQ ID NO: 10 - DNA, 450 bp, Bos sp.
gataagcaagcaaaatccccacagctagagcctggcaacaccgaccaggaaagtgaggaacagcggcaattccggaatattttcaggcagatagcaggcgatgacatggagatctgcgcagatgagctcaagaacgtccttaacagagttgtgaacaaacataaggacctgaagacacaaggcttcacgctggagtcctgccgtagcatgatygctctcatggacacagatggctctgggagactgaacctgcaagagtttcatcacctctggaagaagattaagacgtggcagaaaattttcaaacactatgacacagaccaatctggcaccatcaacagctacgagatgcgcaatgcagtcaaagatgcaggcttccacctcaacaaccagctctacgatatcattaccatgcgctatgcagacaagtacatgaatattgacttcgac

SEQ ID NO: 11 - DNA, 450 bp, Bos sp.
gataagcaagcaaaatccccacagctagagcctggcaacaccgaccaggaaagtgaggaacagcggcaattccggaatattttcaggcagatagcaggcgatgacatggagatctgcgcagatgagctcaagaacgtccttaacagagttgtgaacaaacataaggacctgaagacacaaggcttcacgctggagtcctgccgtagcatgattgctctcatggacacagwtggctctgggagactgaacctgcaagagtttcatcacctctggaagaagattaagacgtggcagaaaattttcaaacactatgacacagaccaatctggcaccatcaacagctacgagatgcgcaatgcagtcaaagatgcaggcttccacctcaacaaccagctctacgatatcattaccatgcgctatgcagacaagtacatgaatattgacttcgac

SEQ ID NO: 12 - DNA, 450 bp, Bos sp.
gataagcaagcaaaatccccacagctagagcctggcaacaccgaccaggaaagtgaggaacagcggcaattccggaatattttcaggcagatagcaggcgatgacatggagatctgcgcagatgagctcaagaacgtccttaacagagttgtgaacaaacataaggacctgaagacacaaggcttcacgctggagtcctgccgtagcatgattgctctcatggacacagatggctctgggrgactgaacctgcaagagtttcatcacctctggaagaagattaagacgtggcagaaaattttcaaacactatgacacagaccaatctggcaccatcaacagctacgagatgcgcaatgcagtcaaagatgcaggcttccacctcaacaaccagctctacgatatcattaccatgcgctatgcagacaagtacatgaatattgacttcgac

SEQ ID NO: 13 - DNA, 450 bp, Bos sp.
ccgtagcatgattgctctcatggacacagatggctctgggagactgaacctgcaagagtttcatcacctctggaagaagattaagacgtggcagaaaattttcaaacactatgacacagaccaatctggcaccatcaacagctacgagatgcgcaatgcagtcaaagatgcaggcttccacctcaacaaccagctctacgatatcattaccatgcgctatgcagacaagtacatgaatattgacttygacagtttcatctgctgctttgtcaggctggagggcatgttcagagctttcaatgcatttgacaaggatggggacggtatcatcaaactcaatgttctcgagtggctgcagctcaccatgtatgcctgaaccaagctggccacatcgaaggcatggaggatcactcaggatttcaatttcacccaacagagctagtcacttac

SEQ ID NO: 14 - DNA, 23 bp, Artificial
  Primer sequence
ggacggtatcatcaaactcaatg

SEQ ID NO: 15 - DNA, 23 bp, Artificial
  Primer sequence
agactccaagcaaagctctcact

SEQ ID NO: 16 - DNA, 14 bp, Artificial
  Primer sequence
cttccgtgtctggc

SEQ ID NO: 17 - DNA, 15 bp, Artificial
  Primer sequence
tcttccctgtctggc

SEQ ID NO: 18 - DNA, 22 bp, Artificial
  Primer sequence
agcctcagttatggctttatgc

SEQ ID NO: 19 - DNA, 23 bp, Artificial
  Primer sequence
agatcagaatgcacaatgagaca

SEQ ID NO: 20 - DNA, 13 bp, Artificial
  Primer sequence
tgagcccatgtcc

SEQ ID NO: 21 - DNA, 13 bp, Artificial
  Primer sequence
tgagcccaggtcc

SEQ ID NO: 22 - DNA, 18 bp, Artificial
  Primer sequence
atgccgaccgtcattagc

SEQ ID NO: 23 - DNA, 22 bp, Artificial
  Primer sequence
gagtagatgccacttgggtttc

SEQ ID NO: 24 - DNA, 15 bp, Artificial
  Primer sequence
tacacgctcacatgc

SEQ ID NO: 25 - DNA, 15 bp, Artificial
  Primer sequence
acactctcacatgct

SEQ ID NO: 26 - DNA, 18 bp, Artificial
  Primer sequence
attgcatggcctcctgac

SEQ ID NO: 27 - DNA, 21 bp, Artificial
  Primer sequence
ctccagaacacctctggactg

(19) United States
(12) Patent Application Publication
(10) Pub. No.: US 2009/0148844 A1
(43) Pub. Date: Jun. 11, 2009

(54) DNA MARKER FOR MEAT TENDERNESS IN CATTLE

(75) Inventor: William Barendse, Queensland (AU)

Correspondence Address: SUGHRUE MION, PLLC, 2100 PENNSYLVANIA AVENUE, N.W., SUITE 800, WASHINGTON, DC, 20037, US

(73) Assignees: COMMONWEALTH SCIENTIFIC INDUSTRIAL AND RESEARCH ORGANISATION, Campbell, Act (AU); THE STATE OF QUEENSLAND THROUGH ITS DEPT., OF PRIMARY INDUSTRIES AND FISHERIES, Queensland (AU); THE UNIVERSITY OF NEW ENGLAND, New South Wales (AU); DEPT. OF PRIMARY INDUSTRIES FOR AND ON BEHALF OF THE STATE OF NEW SOUTH WALES, New South Wales (AU); MEAT & LIVESTOCK AUSTRALIA LIMITED, New South Wales (AU)

(21) Appl. No.: 12/093,200

(22) PCT Filed: Nov. 9, 2006
(86) PCT No.: PCT/AU2006/001667

§ 371 Date: Sep. 24, 2008

(30) Foreign Application Priority Data
Nov. 11, 2005 (AU) 2005906261

(51) Int. Cl. C12Q 1/68; C07H 21/04

(52) U.S. Cl. 435/6; 536/23.5; 536/24.3

(57) ABSTRACT

A method for assessing the tenderness of meat from an animal, comprising the step of testing the animal for a genetic marker in the calpain3 (CAPN3) gene associated with Warner-Bratzler peak force variation or for a genetic marker located other than in CAPN3 which shows allelic association therewith.

### TECHNICAL FIELD

The present invention is concerned with genetic markers for meat tenderness in animals, and with methods and oligonucleotide probes for assessing meat tenderness in said animals, and a kit for this purpose. The invention is useful for the selection of animals which show desirable traits in meat tenderness either for breeding or to select animals destined to be slaughtered for food.

### BACKGROUND ART

Meat tenderness is an important issue for consumers, and one which can influence demand sufficiently for an especially tender meat to command a premium price in the marketplace. The physiological change in muscle structure during the post-mortem period is complex but clearly seems to be at least one factor in meat tenderness. It has been theorised that an endogenous, calcium-dependent proteinase, calpain, initiates in vivo muscle protein degradation. In particular, calpain is believed to be responsible for the breakdown of myofibril protein, which is closely related to meat tenderness. Two isoforms of calpain have been isolated, and these are referred to as μ-calpain and m-calpain. Each of these consists of an 80 kDa catalytic sub-unit, which are products of separate genes, and a 30 kDa regulatory sub-unit, which is identical in both enzymes. Both μ-calpain and m-calpain have been shown to degrade most of the fibrular protein, excluding, however, actin and myosin. Accordingly these classical calpains and the molecule which regulates their activity, calpastatin, play a key role in the tenderisation process that occurs during post-mortem storage of meat under refrigeration.

In addition to the classical calpains, cloning and sequencing DNA has led to identification of a number of calpain-like genes in different organisms or in specific tissues. With few exceptions the proteins encoded by the expressed mRNAs have not been isolated from tissues, so very little is known about the catalytic or other properties of the proteins encoded by these genes. At least twelve different mRNAs or genes encoding polypeptides with sequence homology to the calpains have been identified in vertebrates, and several are expressed principally in skeletal or smooth muscle cells. In particular, calpain 3 (CAPN3) is expressed principally in skeletal muscle and has recently been shown (Ilian et al., 2004) to play a role in post-mortem tenderisation of meat via the proteolysis of specific muscle structural proteins such as nebulin.

### SUMMARY OF THE INVENTION

The present inventors have now shown an association between genetic variation at CAPN3 and meat tenderness.

Accordingly, in a first aspect of the present invention there is provided a method for assessing the tenderness of meat from an animal, comprising the step of testing the animal for a genetic marker in the calpain3 (CAPN3) gene associated with Warner-Bratzler peak force variation or for a genetic marker located other than in CAPN3 which shows allelic association therewith.

It will be appreciated that the test may be a positive test for the presence of the polymorphism associated with low Warner-Bratzler peak-force or a negative test confirming the absence of the polymorphism. Either test allows the tester to reach the same conclusion concerning the meat tenderness characteristics of a beast. Thus a beast identified as having the potential for more tender beef could be used for breeding to improve the herd. Alternatively this test could simply mark the beast as more suitable for slaughter if higher quality, more tender beef is desired.

In an embodiment, the polymorphism is a single nucleotide polymorphism (SNP) identified in Table 8 or 11.

It was not previously known that variation between the DNA of individuals at CAPN3 affected meat tenderness. However, given the identification of a DNA variance at CAPN3 associated with increased meat tenderness it follows that other polymorphisms in and around the calpain 3 gene could be used as tools to select for meat tenderness. Where there has been a relatively recent reduction in population size for a species, particular haplotypes of individuals will be relatively over-represented. If insufficient time has elapsed to cause allelic association to decay, there will be linkage disequilibrium even for alleles which are far apart. Livestock species such as cattle have been domesticated from a relatively small pool of wild ancestors in recent times and therefore in these species allelic association is found between alleles that may be remote physically (Farnir et al. 2000; McRae et al. 2002; Tenesa et al. 2003; Nsengimana et al. 2004). Thus it follows that regions of genetic variation that are outside of CAPN3 will also show allelic association with the polymorphisms in that gene, and therefore will be suitable genetic markers for the characteristic of peak-force variation. Hence, these polymorphisms may also be used to assess meat tenderness. In effect, these polymorphisms serve as markers for the same causative genetic difference. Further polymorphisms, either known or unknown, may be assayed using techniques such as TAQMAN assay, primer extension and oligonucleotide ligation assays. Furthermore, the design of specific primers to amplify a particular DNA fragment is well within the capability of the person skilled in the art; therefore any region of interest of CAPN3 may be amplified with appropriate primers. Equally, primers other than those specifically disclosed above may be used to amplify the polymorphisms identified in Table 8 or 11.

It will be appreciated that the association of an allele or genotype with increased value will often apply across breeds and families within breeds. However, a particular allele or genotype may not always be associated with increased values across breeds; in one breed the allele or genotype might be associated with an increased value but in another breed it might be associated with decreased value or not be associated with difference in value. The person skilled in the art will be able to establish the direction of the association following the teachings herein.

In an embodiment haplotypes consisting of the CAPN3:C.53 T>G-T, CAPN3JK-G and CAPN3:C.2443-103G>C-C allele as set forth in Table 1 are associated with improved meat tenderness in Brahman cattle.

In an embodiment haplotypes consisting of the CAPN3:C.53 T>G-G, CAPN3JK-G and CAPN3:C.2443-103G>C-G allele are associated with improved meat tenderness in Belmont Red Cattle.

In an embodiment the polymorphism and flanking sequence of comprises the nucleotide sequence set forth in any one of SEQ ID Nos: 1 to 3 and 5 to 15.

According to yet another aspect of the present invention there is provided a method for selecting an animal likely to yield meat of improved tenderness, comprising the steps of:
(1) testing the animal for the presence of a genetic marker in CAPN3 associated with low peak-force or for a genetic marker located other than in CAPN3 which shows allelic association therewith; and
(2) selecting animals in said genetic marker is present.

Advantageously, in order to assess the tenderness of meat from an animal and/or to select an animal likely to yield meat with improved tenderness, testing may comprise the steps of:

1) obtaining a biological sample from the animal;

2) extracting DNA from the sample;

3) amplifying DNA from CAPN3 and/or from regions surrounding CAPN3 in order to amplify instances of genetic variation which show allelic association to polymorphisms at CAPN3; and

4) identifying the allele present in the amplified DNA.

In a still further aspect the invention provides an oligonucleotide probe for amplification of a genetic marker associated with low Warner-Bratzler peak-force, said genetic marker being either a polymorphism in the calpain3 (CAPN3) gene associated with Warner-Bratzler peak force variation or for a genetic marker located other than in CAPN3 which shows allelic association therewith.

In a still further aspect the present invention provides a kit for use in assessing the tenderness of meat from an animal and/or selecting an animal likely to yield meat of improved tenderness, comprising oligonucleotide probes for amplification of at least one genetic marker for meat tenderness as described above.

This specification contains nucleotide and amino acid sequence information prepared using PatentIn Version 3.3. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g., SEQ ID NO: 1 refers to the sequence in the sequence listing designated as <210>1 and the sequence information immediately follows the identifier <400>1). In the sequences the symbols Y, R, M, K, S and W have been used to indicate the polymorphism. Thus the symbol “M” represents an A/T polymorphism, and so on. The sequence flanking the polymorphism is derived from publicly available sequence information. The present invention is not restricted to detection of the entire nucleotide sequence or in any way restricted to use of the entire nucleotide sequence. This information is presented to assist in the design of oligonucleotide premiers and probes, but the person skilled in the art will recognise that such sequence may contain errors and will adjust their design accordingly.

The designation of nucleotide residues referred to herein are those recommended by the IUPAC-IUB Biochemical Nomenclature Commission, wherein A represents Adenine, C represents Cytosine, G represents Guanine, T represents Thymine, Y represents a pyrimidine residue, R presents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Throughout this specification, unless specifically states otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein any range of numerals includes all within the range and the term “at least one” means one, two, three, four, etc. up to the possible maximum. Therefore a reference to one or more SNPs includes one SNP or any number of SNPs from two to the total number of SNPs set forth herein. The SNPs set forth herein can be used alone or in any combination, therefore the invention envisages detection of any of the possible combinations of SNPs set forth herein.

As used herein the term “cow’ is used to refer to an individual animal without an intention to limit by gender and should not be taken to do so unless it is necessary from the context to infer that a female animal is referred to. The term should also be taken to encompass a young animal of either gender.

The present invention is not be limited in scope by the specific embodiments described herein, which are intended for the purpose of exemplification only. Functionally-equivalent products, compositions and methods are clearly within the scope of the invention, as described herein.

The present invention may be performed following the description herein without undue experimentation using, unless otherwise indicated, conventional techniques of molecular biology. Such procedures are described in various publications referred to throughout the specification, and the content of each such publication is incorporated herein by reference.

Throughout this specification and the claims, the words “comprise”, “comprises” and “comprising” are used in a non-exclusive sense, except where the context requires otherwise.

It will be clearly understood that, although a number of prior art publications are referred to herein, this reference does not constitute an admission that any of these documents forms part of the common general knowledge in the art, in Australia or in any other country.

Those skilled in the art will appreciate that the invention described herein is susceptible to variations and modifications other than those specifically described. It is to be understood that the invention includes all such variations and compounds referred to or indicated in this specification, individually or collectively, and any and all combinations or any two or more of said steps or features.

### DETAILED DESCRIPTION OF THE INVENTION

The methods of the invention allow for the management of bovine animals including selection of animals for breeding or cloning. The methods allow the identification of animals with favourable LDPF characteristics.

The principal commercial bovine animals are cattle, and there are many breeds of cattle. In an embodiment the cow is a breed (or cross) selected not exclusively from the group consisting of Angus, Ankole-Watusi, Ayrshire, Bazadaise, Beefalo, Beefmaster, Belgian Blue, Belmont Red, Blonde d'Aquitaine, Bonsmara, Braford, Brahman, Brahmousin, Brangus, Braunvieh, British White, American Brown Swiss, BueLingo, Charolais, Chianina, Corriente, American Devon, Dexters, Droughtmaster, Galloway, Gelbvieh, Guernsey, Hereford, Highland, Holstein, Jersey, Limousin, Lowline, Maine-Anjou, Marchigiana, Milking Shorthorn, Montebeliarde, Murray Grey, Normande, Parthenaise, Piedmontese, Pinzgauer, Romagnola, Salers, Salorn, Santa Gertrudis, Shetland, Shorthorn, Simmental, South Devon, Tarentaise, Texas Longhorn and Wagyu, most particularly Angus, Shorthorn, Hereford, Murray Grey, Brahman, Belmont Red and Santa Gertrudis.

Single nucleotide polymorphisms are allelic variants that occur in a population where a single nucleotide difference is present at a locus. The method of the invention can involve detection of one single nucleotide polymorphism or more than one single nucleotide polymorphism, and can involve detection of single nucleotide polymorphisms which form or are a part of a haplotype which, as used herein, refers to groupings of two or more single nucleotide polymorphisms that are physically present in the same chromosome and tend to be inherited together except when recombination occurs. Methods for identifying haplotype alleles in nucleic acid samples are known to the person skilled in the art. This is from methods for haplotyping are described in WO 2005/040400, the contents of which are incorporated herein by reference.

A preferred sample for performing the method of the invention is a readily accessible sample that comprises genomic DNA. For example, genetic testing of cattle is often performed using a hair follicle, for example, isolated from the tail of an animal to be tested.

Other examples of readily accessible samples include, for example, bodily fluids or an extract thereof or a fraction thereof. For example, a readily accessible bodily fluid includes, for example, whole blood, saliva, semen or urine.

In another embodiment, a biological sample comprises a cell or cell extract or mixture thereof derived from a tissue such as, for example, skin.

Preferably, a biological sample has been isolated or derived previously from a subject by, for example, surgery, or using a syringe or swab.

Cell preparations or nucleic acid preparation derived from such tissues or cells are not to be excluded. The sample can be prepared on a solid matrix for histological analyses, or alternatively, in a suitable solution such as, for example, an extraction buffer or suspension buffer, and the present invention clearly extends to the testing of biological solutions thus prepared.

Analysis of the sample may be carried out by a number of methods. The present invention has identified a number of SNPs associated with traits in bovine animals, and subsequently detecting the presence or absence of the favourable allelic form of each SNP, or a plurality of these SNPs can be done using methods known in the art. Such methods may employ one or more oligonucleotide probes or primers including, for example, an amplification primer pair that selectively hybridize to a target polynucleotide which comprises a part or all of the sequence set forth in any one of SEQ ID Nos:1 to 15. Oligonucleotide probes useful in an embodiment of the invention comprise an oligonucleotide which is complementary to and spans a portion of the polynucleotide including the SNP in question. Therefore, the presence of a specific nucleotide at the position (i.e. one of the allelic forms of the SNP) is detected by the ability or otherwise for the probe to hybridize. Such a method can further include contacting the target polynucleotide and hybridized oligonucleotide with an endonuclease and detecting the presence or absence of a cleavage product of the probe.

An oligonucleotide ligation assay also can be used to identify a nucleotide occurrence at a polymorphic position, wherein a pair of probes that selectively hybridize upstream and adjacent to and downstream and adjacent to the site of the SNP are prepared, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the “dideoxy-mediated chain termination method,” also known as the “Sanger Method” (Sanger, F., et al. J. Molec. Biol. 94:441 (1975); Prober et al. Science 238:336-340 (1987)) and the “chemical degradation method,” “also known as the “Maxam-Gilbert method” (Maxam, A. M., et al. Proc. Natl, Acad. Sci (U.S.A.) 74:560 (1977)), the contents of which are herein incorporated by reference to determine the nucleotide occurrence at the SNP loci.

As will be apparent to the person skilled in the art, the specific probe or primer used in an assay of the present invention will depend upon the assay format used. Methods of designing probes and/or primers for example, PCR or hybridization are known in the art and described, for example, in Dieffenbach and Dveksler (Eds) (In: PCR Primer: A Laboratory Manual, Cold Spring Harbour Laboratories, NY, 1995) and more recently in P.-Y. Kwok (Ed) (In: Methods in Molecular Biology Vol 212 Human Press, Totowa N.J., 2003). The various categories of polymorphism have been systematized and the various methods used to detect them have been thoroughly overviewed (Barendse and Fries 1999). In short, there are only two kinds of polymorphism, those due to changes of DNA bases and those due to insertion and deletion of bases. Furthermore, the detection of these polymorphisms uses essentially the same technology and it is a rare technique that can be used for only one or the other of these kinds of polymorphism. Polymorphisms can also be divided into those that are in or near a sequence that is transcribed into RNA (type I polymorphisms) and those that are in DNA that is never translated into RNA (type II polymorphisms). However, all of these polymorphisms can be detected using the same kinds of methods. The methods for detecting DNA polymorphisms revolve around 3 major aspects, not all of which are used in every detection method, and some methods use techniques that are not one of these 3 major kinds. The point is that there are a large and ever increasing range of methods for detecting polymorphisms so it is not possible to be prescriptive about how the polymorphism should be detected, rather, it is the DNA sequence which lends itself to one or the other method of detection. Most of these methods are highly dependent upon the polymerase chain reaction (PCR), although it is possible to detect sequence differences easily without using the PCR. The three technologies are 1) separation of DNA by size or by composition, 2) oligonucleotide hybridisation to recognise specific DNA sequences, and 3) DNA visualisation. The DNA separation may be performed on solid matrices but may be performed in liquid matrices. The recognition of DNA sequence is usually performed by oligonucleotides of predefined sequence but may be performed enzymatically since some enzymes recognise specific DNA sequence motifs. DNA visualisation can be performed directly on the DNA which binds to some elements such as silver when it is visible in ordinary light, it may fluoresce under ultra violet light when it is bound to some molecules such as ethidium bromide, or it may be visualised through autoradiography when radioactive nucleotides are incorporated into the sequence. More usually, the DNA is visualised when it is bound to a DNA oligonucleotide which has a previously attached reporter molecule which may then be detected after laser excitation. Many methods depend on reporting the result of a specific reaction on the DNA, and may not even detect the DNA itself but remnants of the successful detection. These descriptions are merely to indicate the wide range and the many possible permutations of DNA detection, and do not exclude methods that have not been specifically referred to.

Some of the methods that might be used to detect the polymorphisms are described below, but they are not the only possible methods. While the specific hybridization of the probe or primer or other method for detecting variability to any nucleic acid can be predicted using well known rules, the probe or primer may not be unique if it is designed to bind to repetitive DNA sequence or to sequence common to members of a gene family, and so precautionary screening of probes and primers should be performed using, for example, BLAST against the cow and other genomes. In many cases this will not be sufficient and the adequacy of probes or primers may need to be confirmed empirically using methods known in the art. This same proviso will apply to all methods of detecting DNA that uses short probes and primers as would be appreciated by anyone skilled in the art.

The following is a list of some of the more useful current high throughput methods of detecting polymorphisms in cattle, but these should not be taken as an exhaustive list or be used to exclude new methods yet to be developed where they are essentially being used to identify the underlying DNA sequence. In that regard, if the DNA sequence has been transcribed to RNA and the RNA is tested for variation, or if the RNA has been translated to protein and the protein is tested for variation, these RNA and protein detection methods will also be methods of detecting the underlying DNA sequence. AS indicated above, sometimes the detection method reports the result of a successful reaction without directly detecting the DNA molecule, and obviously this would apply for RNA and protein as well.

Some of the useful DNA based methods of detecting polymorphisms are the Taqman assay (LIVAK 2003), which uses competitive hybridisation of probes specific for the alternative DNA sequences and where a successful reaction is detected through the liberation of a reporter dye, the SNPlex assay (Applied Biosystems Incorporated, Foster City, Calif.) which uses the oligonucleotide ligation assay and where a successful reaction is reported via Zipchute probes that are separated on a capillary DNA sequencer, high throughput molecular inversion probes associated with generic microarray technologies (HARDENBOL et al. 2003; HARDENBOL et al. 2005), the MASSextend (STORM et al. 2002) or generic primer extension technologies (CHEN et al. 1997) which use mass spectrometry or laser fluorescence of the probe modified by an enzyme reaction respectively.

The present invention also relates to kits which can be used in the above described methods. Such kits typically contain an oligonucleotide probe, primer, or primer pair, or combinations thereof, depending upon the method to be employed. Such oligonucleotides are useful, for example, to identify a SNP as set forth herein. In addition, the kit may contain a control comprising oligonucleotides corresponding to the nucleotide sequence of the non-desired allelic form. In addition, the kit may contain reagents for performing a method of the invention such as buffers, detectable labels, one or more polymerases, which can be useful for a method that includes a primer extension or amplification procedure, and are nucleases for digesting hybridization products or a ligase which can be useful for performing an oligonucleotide ligation assay. The primers or probes can be included in the kit in labelled form.

The kit may also include instructions for use.

As well as management of the individual animal, the present invention allows for selection of animals for breeding programs. Thus a herd may be developed with desirable LDPF characteristics so as to have enhanced longissimus dorsi peak force. The method involves selecting animals with desirable characteristics and using them in a breeding program. The progeny of the mating of selected parents are likely to exhibit the trait, thus creating a line of animals with specific characteristics. The progeny can then be used to breed and so on in order to continue the line, which may be monitored for purity using the original SNP markers.

Furthermore, the method of the invention allows for in vitro methods of producing animals. In general terms the method involves identification of one or more SNPs as set forth in SEQ ID Nos: 1 to 15 in a bovine animal, isolating a progenitor cell from the animal and generating an animal from the progenitor cell. Methods of cloning bovine animals are well known to the person skilled in the art. For methods involved in cloning of cattle known methods may be used directly. As set forth, for example, in S. M. Willadsen “Cloning of sheep and cow embryos” Genome 31:956 (1989), the contents of which are incorporated herein by reference.

In an embodiment, following development of an embryo, one or more cells is/are isolated therefrom and screened as described above. An embryo having or likely to have possess the desired trait is then selected and implanted into a suitable recipient. In this manner, animals having or likely to have improved meat tenderness are produced.

In an embodiment the selected animals are used to produce offspring using in vitro fertilization. In this process, ova are harvested from a cow comprising one or more SNP of the invention by, for example, transvaginal ovum pick-up (OPU) or by laparoscopic aspiration. The recovered ovum is then matured prior to fertilization. Zygotes are then cultured for a time and under conditions suitable for embryo development. For example, zygotes are cultured in a ligated oviduct of a temporary recipient (sheep or rabbit). Alternatively, zygotes are co-cultured in vitro with somatic cells (e.g., oviduct epithelial cells, granulosa cells, etc) in a defined medium. Alternatively, zygotes are cultured in vitro in a simple medium such as synthetic oviductal fluid without any somatic cell support.

The method is amenable to screening embryos produced using any assisted breeding technology and/or for screening embryos produced using an ovum and/or sperm from an animal that has not been screened using the method of the invention.

### Modes for Performing the Invention

A gene for beef tenderness was located to bovine chromosome 10 in the CBX experiment in the period 1996-1999. One sire, CBX 7, shows suggestive evidence for a QTL affecting LD compression (lod=2.27), the peak located between the DNA markers CSRM60 and DIK20 (Hetzel and Davis, 1999). This region of bovine chromosome 10 is now known to include genes from Human chromosome 15. An examination of human chromosome 15 revealed the presence of the CAPN3 gene, which has also been mapped to bovine chromosome 10 (Normeman and Koohmaraie 1999). Since this gene has become implicated in meat tenderness in studies in sheep (Ilian et al., 2001, 2004), it was selected as the primary positional candidate for the effect. SNP were sought in the gene and were tested for effects on meat tenderness on a diverse range of cattle. A statistically significant effect of genetic substitution at the CAPN3 gene was found on peak force measurements of the LD (longissimus dorsi) muscle.

Cattle were chosen from the CRC DNA Bank to have as diverse a genetic and phenotypic background as possible. Information stored in the CRC Database was used to select animals. Animals of extremes of peak-force were selected, although animals with peak-force measures above 12 were excluded since they might have confounded peak-force measurements. In essence, the procedure was to select cattle in each cohort which were of phenotypic extreme measures, to ensure that no sire was represented by a cluster of offspring, that all markets and finishing regimes were included in each extreme, so that extremes were not biased by being representative of a particular market or finishing regime. A total of 246 samples were obtained, and were the combined samples used in the CAST experiments (Barendse, 2001 WO02064820, PCT/AU02/00122) (Tables 1 and 2 combined).

### EXAMPLE 1

These DNA samples were genotyped for the CAPN3X3 G266C (Calpain 3 intron 24 and 3′UTR G->C base 266) DNA fragment. This polymorphism was obtained by sequencing a panel of 12 animals including 2 of zebu ancestry for a PCR fragment including intron 24 and the 3′UTR (untranslated region) using the following primers:

| | |
|---|---|
| CAPN3X3U1 GGACGGTATCATCAAACTCAATG | (SEQ ID NO:14) |
| CAPN3X3D1 AGACTCCAAGCAAAGCTCTCACT | (SEQ ID NO:15) |

A SNP was discovered in this fragment at base 266 as per the following DNA sequence:

>CAPN3X3|Bta|Contig1 good seq 17-678 snp 1 zebu
opp hoM g266c

(SEQ ID NO:1)

TTTTTGGGAAAACATCAAACTCAATGTTCTCGAGGTAAAGCCTAACAGTA
CACTCCCCCCGCACTTTAAAATTTGAGGTGGAGGGATGGGATTGGATGGG
ATGGGAAAGGAACATAAAGGGAAGCTCAGAGCTCAGCAGGATGTGGTCAT
GGGGAACGCATAGTGGGGGAAGGCTCTAAGGAAGCCTCAGTTATGGCTTT
ATGCTGGAAAATCTGTACTTTCTAGGGGAGATCTGTGGGAAGGATGCTTC
TCAGGAAATCTTCCgTGTCTGGCATGCTGGGACTGGCCTTTTCTTCTACG
TTTTAGATATGTAGGGCTTGGCTCTTTTGTCTCATTGTGCATTCTGATCT
TGGGACTTCCTCTTGCAGTGGCTGCAGCTCACCATGTATGCCTGAACCAA
GCTGGCCACATCGAAGGCATGGAGGATCACTCAGGATTTCAATTTCACCC
AACAGAGCTAGTCACTTACCTCAAAGGACACAGTTGCTGCACCCCCAGGA
AGCCTCCAGGCACCTCATCAGTCTAGTTCCTCCTCTACTCACTTCCCTCC
CAGCCTTAGGGCATCTGTCCATTTGTCAGGCCCAGCCTGACCCTTCAGTT
AAGGAATGGGGAAGGGAGAGCTGTGTTGTCCCTGTGCCACATGGAGCCAA
GTGCCTCTGTCTGCTTCCGCTAGCCACGACTACATTTA

This SNP was assayed using the following pair of Taqman probes:

| | |
|---|---|
| CAPN3X3-266G 6FAM-CTTCCGTGTCTGGC | (SEQ ID NO:16) |
| CAPN3X3-266C VIC-TCTTCCCTGTCTGGC | (SEQ ID NO:17) |

PCR primers to amplify the region around the SNP consist of combinations of the following up and down primers:

(SEQ ID NO:18)

CAPN3X3G266CU2 AGCCTCAGTTATGGCTTTATGC

(SEQ ID NO:19)

CAPN3X3G266CD1 AGATCAGAATGCACAATGAGACA

The PCR occurred in 10 μL, contained 50 ng DNA, 0.2 μM per probe and 0.9 μM per primer, in ABI Taqman Universal PCR Mastermix, for 40 cycles, annealing/extension at 60 C for 60 seconds, using an ABI 7900HT.

The genotypes were analysed using generalised linear models (GLM) following the equation peakforce=1+fixed+random effects+genotypes+error implemented via the S-PLUS software. Fixed effects that were considered were breed, market (Domestic, Korea, Japan), cohort, finish (pasture v grain, north v south) and the random effect of sire. Some models did not include sire, since the samples were chosen to have as few animals per sire as possible. The size of the effects associated with genotype was estimated by the comparison of the sum of squares of Analysis of Variance models with and without the marker genotypes. Plots of raw peakforce values against Calpain 3 genotypes were constructed.

The full data set shows a statistically significant association between genotype and LD peak force when sire is included in the model. Neither sire nor breed of origin was statistically significant consistent with the scheme of sampling across a wide range of breeds and sires and using animals of extreme phenotype as can be seen the following table.

TABLE 1
Analysis of Deviance Table (CAPN3 for all
breeds including sire as a random variable)
Gaussian model
Response: ldpf
Terms added sequentially (first to last)

| | Df Deviance | | Df Resid. | | | |
|---|---|---|---|---|---|---|
| | Resid. | | Dev | | F Value | Pr(F) |
| NULL | | | 241 | 1135.673 | | |
| market | 2 | 114.7534 | 239 | 1020.919 | 21.92078 | 0.0000000 |
| cohort | 26 | 353.6020 | 213 | 667.317 | 5.19591 | 0.0000000 |
| finish | 3 | 67.5197 | 210 | 599.798 | 8.59863 | 0.0000659 |
| breed | 4 | 9.2969 | 206 | 590.501 | 0.88797 | 0.4760605 |
| sireid | 137 | 396.8467 | 69 | 193.654 | 1.10668 | 0.3254380 |
| genotype | 2 | 18.2843 | 67 | 175.370 | 3.49276 | 0.0360653 |

A similar association was found when the effect of sire was excluded from the analysis as can be seen in the following table.

TABLE 2
Analysis of Deviance Table
Gaussian model
Response: ldpf
Terms added sequentially (first to last)

| | Df Deviance | | Df Resid. | | | |
|---|---|---|---|---|---|---|
| | Resid. | | Dev | | F Value | Pr(F) |
| NULL | | | 241 | 1135.673 | | |
| market | 2 | 114.7534 | 239 | 1020.919 | 20.63407 | 0.0000000 |
| cohort | 26 | 353.6020 | 213 | 667.317 | 4.89092 | 0.0000000 |
| finish | 3 | 67.5197 | 210 | 599.798 | 8.09391 | 0.0000404 |
| breed | 4 | 9.2969 | 206 | 590.501 | 0.83584 | 0.5038044 |
| genotype | 2 | 23.2422 | 204 | 567.258 | 4.17924 | 0.0166409 |

The average deviation for the cc genotype is +0.96 kg PF, for the cg genotype is +0.024 kg PF and for the gg genotype is −0.98 kg PF.

However, the frequency of the c allele is extremely low in the taurine breeds and so the analysis was redone using only the Brahman, Belmont Red and Santa Gertrudis animals. Allele frequencies are shown below.

TABLE 3

| Breed | cc | cg | gg | p(c) |
|---|---|---|---|---|
| Angus | 0 | 1 | 43 | 0.01 |
| Brahman | 1 | 9 | 33 | 0.13 |
| Belmont Red | 1 | 10 | 26 | 0.16 |
| Hereford | 0 | 0 | 39 | 0.00 |
| Santa Gertrudis | 0 | 7 | 30 | 0.10 |
| Shorthorn | 0 | 0 | 42 | 0.00 |

Again, there is a statistically significant association between genotype and meat tenderness.

TABLE 4
Analysis of Deviance Table
Gaussian model
Response: ldpf
Terms added sequentially (first to last)

| | Df Deviance | | Df Resid. | | | |
|---|---|---|---|---|---|---|
| | Resid. | | Dev | | F Value | Pr(F) |
| NULL | | | 116 | 503.4443 | | |
| market | 2 | 34.6401 | 114 | 468.8042 | 6.828450 | 0.0017177 |
| cohort | 16 | 180.6520 | 98 | 288.1522 | 4.451392 | 0.0000020 |
| finish | 2 | 27.7125 | 96 | 260.4398 | 5.462838 | 0.0057299 |
| breed | 2 | 7.6218 | 94 | 252.8180 | 1.502455 | 0.2279932 |
| genotype | 2 | 19.4642 | 92 | 233.3537 | 3.836900 | 0.0250907 |

The average deviation for the cc genotype is +0.84 kg PF, for the cg genotype is +0.062 kg PF and for the gg genotype is −0.90 kg PF. A similar result is found when sire is included in the model as seen below but then the size of effect expands to a massive average deviation for the cc genotype of +2.84 kg PF, for the cg genotype of −0.91 kg PF and for the gg genotype of −1.93 kg PF. These values are probably too large and are probably not accurately estimated.

TABLE 5
Analysis of Deviance Table
Gaussian model
Response: ldpf
Terms added sequentially (first to last)

| | Df Deviance | | Df Resid. | | | |
|---|---|---|---|---|---|---|
| | Resid. | | Dev | | F Value | Pr(F) |
| NULL | | | 116 | 503.4443 | | |
| market | 2 | 34.6401 | 114 | 468.8042 | 8.418921 | 0.0013715 |
| cohort | 16 | 180.6520 | 98 | 288.1522 | 5.488204 | 0.0000456 |
| finish | 2 | 27.7125 | 96 | 260.4398 | 6.735234 | 0.0040914 |
| breed | 2 | 7.6218 | 94 | 252.8180 | 1.852404 | 0.1755742 |
| sireid | 64 | 178.7883 | 30 | 74.0296 | 1.357896 | 0.1874672 |
| genotype | 2 | 16.4259 | 28 | 57.6037 | 3.992150 | 0.0298284 |

TABLE 6
Characteristics of the first Cattle Sample

| | |
|---|---|
| Total: | 169 |
| | 83 high peak force |
| | 86 low peak force |
| Breeds: | 29 Santa Gertrudis |
| | 25 Hereford |
| | 26 Angus |
| | 27 Belmont Red |
| | 31 Brahman |
| | 31 Shorthorn |
| Regions: | 38 Pasture South |
| | 28 Pasture North |
| | 57 Grain South |
| | 41 Grain North |
| Markets: | 72 Korean |
| | 67 Domestic |
| | 25 Japanese |
| Cohorts: | 27 Cohorts |
| | Median: 5 steers per cohort |
| | bottom quartile: 2 steers per cohort |
| | top quartile: 9 steers per cohort |
| Sires: | 112 sires |
| | Median: 1 steer per sire |
| | bottom quartile: 1 steer per sire |
| | top quartile: 2 steers per sire |

TABLE 7
Characteristics of the second sample of 77 animals

| | |
|---|---|
| Total: | 77 |
| | 39 high peak force |
| | 38 low peak force |
| Breeds: | 11 Belmont Red |
| | 11 Hereford |
| | 13 Brahman |
| | 13 Shorthorn |
| | 14 Santa Gertrudis |
| | 15 Angus |
| Regions: | 24 Pasture South |
| | 12 Pasture North |
| | 21 Grain South |
| | 20 Grain North |
| Markets: | 35 Korean |
| | 25 Domestic |
| | 17 Japanese |
| Cohorts: | 22 Cohorts |
| | Median: 3 steers per cohort |
| | bottom quartile: 2 steers per cohort |
| | top quartile: 5 steers per cohort |
| Sires: | 64 sires |
| | Median: 1 animal per sire |
| | bottom quartile: 1 animal per sire |
| | top quartile: 1 animal per sire |

### EXAMPLE 2

The initial associations between CAPN3X3 to meat tenderness were found using a sample of animals of extremes of tenderness. To explore the association further, to measure the effect of allele substitution at the gene, and to determine the breed differences that might be found, additional polymorphisms in the gene were identified, including one affecting the amino acid sequence, and genotyped on more than 2,000 animals of Brahman, Belmont Red and Santa Gertrudis ancestry from the Beef CRC 1 progeny tests DNA bank and database. Animals of pure Taurine ancestry were not examined further since the allele frequencies of all of the SNP are extremely low in purebred Taurine animals.

Two additional SNP were identified and genotyped in addition to CAPN3X3. These are CAPN3E6, a mutation in exon 6, and CAPN3JK, a SNP detected in a fragment containing the 10^{th} and 11^{th} introns. The DNA sequences associated with these SNP are contained in Table 8. These sequences were used to generate Tagman assays and the primers and probes for these are contained in Table 9. The method of determining whether a measured allele or genotype has an increased value compared to others at that locus or more broadly within the gene or genetic region would be familiar to practitioners of the art but will be described briefly. In essence we partition the variance associated with the trait into that due to the Mendelian component associated with the locus under discussion as well as a polygenic component due to shared family. Before this is done, the trait values must be adjusted for fixed environmental and genetic effects, for covariates, and for random genetic effects such as the sire or dam. This is usually performed using a General Linear Mixed Model. Then the genotypes can be compared using at test or a one-way analysis of variance, and the statistical significance can be assessed using permutation tests, particularly where the trait distribution is non-normal. The average effect of allele substitution at the locus is derived from the allele frequency, the difference between homozygotes and the degree of dominance, where α=a[1+k(p−(1−p)] where a is half the difference between homozygotes, p is the allele frequency and k is d/a where d is the difference between the heterozygote and half the distance between the homozygotes. Good starting points for further information on this process are Boerwinkle et al. 1986 Ann. Hum. Genet. 50, 181-194 and Lynch and Walsh, 1997 (Sinauer Associates).

The residual LDPF data were obtained using ASREML using the model:

ldpf˜mu herd kill_group stim_code age cwt ! sireid

in which sireid is a random effect, herd is the herd within breed, kill_group incorporates cohort, finish and market, stim_code is a fixed effect recording the amount of electrical stimulation to the carcass, while age and carcass weight are covariates which affect tenderness.

The association of an allele or genotype with increased value will often apply across breeds and families within breeds. However, a particular allele or genotype may not always be associated with increased value across breeds, in one breed the allele or genotype might be associated with increased value but in another breed it might be associated with decreased value or not be associated with differences in value. The results presented here show both the associations aggregated across the breeds, which represents the alleles or genotypes that will be associated with increased value for most breeds, as well as the associations specifically within breeds. Since the values have been adjusted for the breeds, the associations can be pooled across breeds, and differences in allele frequency in the breeds will not cause the generation of spurious associations due to Simpsons Paradox. However, mixing a breed in which one allele is associated with higher meat tenderness with another breed that has a different allele associated with higher meat tenderness will tend to reduce the size of the effect. This should give an indication where in the gene the causal mutation is likely to be (see below).

We have reported the results for both breeds combined and breeds separated. Firstly, the aggregated statistics give more realistic values of the size of effect because they are based on larger numbers, and secondly, that there are many more breeds in existence than we have access to so it is important to obtain the general association between the allele or the genotype and the trait, which will have applicability in most or all breeds. Clearly, the person skilled in the art will know that some breeds may have different associations between the allele or genotype and the trait due to one of several real biological causes. The first and probably most common is that the measured allele or genotype is not causative, so it is in linkage disequilibrium with the causative allele or genotype. There will be cases where the allele or genotype being measured is in opposite genetic phase to the causative allele or genotype, and this might be reflected in some breed differences. The second is that there may be more than one causative mutation in the gene, with different frequencies in different breeds, hence the measured allele or genotype may show different predictive efficiencies in different breeds and show opposite genetic phase relationships due to complex associations between the measured allele or genotype and the different causative mutations. The third is that a causative mutation in a gene may be affected by genes elsewhere in the genome. These epistatic or background effects have been known for decades, and some of these may have an impact upon the association between the measured allele or genotype and the trait value.

In the associations between CAPN3 and meat tenderness (Table 10), that the CAPN3JK SNP has the largest effect of allele substitution, the only one below the P<0.05 level in the combined data, and the only one in which those breeds that have statistically significant associations to LDPF have the same allele associated with improved tenderness. When we explore this further, we see that all SNP have statistical associations between the Brahman and Belmont Red breed, but none have associations in the Santa Gertrudis breed. Moreover, the association in the CAPN3JK SNP shows the same allele having effects on increasing meat tenderness, while for the CAPN3E6 and CAPN3X3 SNP, the Brahman and Belmont Red have contradictory associations for those SNP, which effectively cancels out the effect of the gene. The association in Santa Gertrudis is not statistically significant in these data so the preferred allele for meat tenderness is irrelevant. The CAPN3JK SNP appears thus to show consistent associations in those breeds in which it has statistically significant associations, where the G allele is associated with improved tenderness. Further SNP near CAPN3JK would be expected to be causal and genotypes at the causal SNP could be predicted using haplotypes.

Since all SNP show associations to meat tenderness in Brahman and Belmont Red animals, haplotypes between these DNA markers might improve prediction since they would encompass the region of the gene that appears to contain the causal mutations. Given the direction of associations in Table 8, for the Brahman for example, haplotypes consisting of the CAPN3E6-T, CAPN3JK-G and CAPN3X3-C allele should improve meat tenderness while in the Belmont Red for example, haplotypes consisting of the CAPN3E6-G, CAPN3JK-G and CAPN3X3-G allele should improve meat tenderness.

These data have been compared to the Calpastatin (CAST) genotypes (Barendse, W. 2001. DNA markers for meat tenderness. Patent WO02064820 (Patent Application PCT/AU02/00122) for these same animals. We see that the overall size of effect of the CAPN3JK genotypes is very similar to that of CAST.

TABLE 8
DNA sequence information for CAPN3 SNP

>CAPN3E6|Bta| exon6 (2nd AA coding exon) AA 34,
bp 100 g100t
ATGCCGACCGTCATTAGCGCCTCTGTGGCCCCACGGACAGGGGCTGAGCC
CA[g/t]GTCCCCAGGGCCCATCGCCCAGGCAGCCCAGGACAAGGGCACC
GAGGCAGGGGGTGGAAACCCAAGTGGCATCTACTCAGCCATCATCAGCCG
CAATTTTCCCATTATTGGGGTGAAAGAGAAGACATTCGAGCAGCTTCACA
AGAAATGTCTGGAAAAGAAGGTTCTTTTTGTGGATCCTGAGTTCCCACCG
GACGAGACCTCCCTGTTTTACAGCCAGAAGTTCCCCATCCAGTTCGTCTG
GAAGAGaCCTCCG
(SEQ ID NO:1)
>CAPN3JK|Bta|Contig1 good bp15-1182, snp zebu opp
hom, 1 taur het bp g642t
TATATAAAAACGGAGAGGACGGAGCTGGGGGCTAACCTCTTCACCATCGG
TTTCGCCATCTACGAGGTGTGCCGTCCCTGACGAGCTTCAGACCGCCTCT
TTAGGGAAGAGGCTTTCAGGGGGCTTCCTGAGGGGTTCCCCAGTTTCCCC
AACATCCAGTGCCCCCCAAAACTACTGATATCAGGCCCACTTGAGTCAGA
ACCTCTTAGAAGTTTGTAACCAGTGGGGGAAGAACCCACCTAGGGGAGGT
GGGGGTGGGGCTGGGGCTGTCCAGGTAGGTACATGGGGCATTAGAGCCCC
TCATTCGCTCTACGATTGCTAGCATGCCCTTTGGCAAGTTTCCATCCCGA
TTAAGACCTGAAATCCACAAAAATCTGAGCACCTTCCTTTTCTGTTTCTC
CCCAAGGTTCCCAAAGAGGTATAGAAGTGGAGCAGCCAGCAGTGTGTGCA
GCGTTACCAGGGTCCTGGGTCCGCCTGGGGCACATCAGGGCACCTCCTGT
GGGGTTTGGGGGCAGGACTGTGATAGGAGAGGGCCTGGCCCACCTTACCT
AACTCCAGAGCAGGTCCTCGGGCCATTGCATGGCCTCCTGACCCCCACCC
CACAACATGCCCTTCCCCTTGCCCACCAGAGACCATTACAC[g/t]CTCA
CATGCTTGCTCACACTCACACACAGAATCACACAGACACATTGTGAGGCT
GATCGCCTTCTTTGGGGGTGGAGGAATCACCTCCCTCAGACCCAGTCCAC
AACCCCCACCCCTGCCTCCCTGGGGTCTTTCCCCAGTCCAGAGGTGTTCT
GGAGCTGAGCAGCAGCAATTCTGGTAAGTGACCCTGAGTGGGGGATGCCA
GACTTGACCTTCACTTCTGGATCACAGCAGAAAAGGAAGAGGATATGGGG
TAGGGAGCTCCAGGGATGTTGAGCTATTCGAGGCCTTGGGAATTGGAAGC
TGGTAGGTTGTGACTATGAAATGAGTGACCAGGGAGTTGGGAAGGAGGAC
CCCTCTGGAGGAGGACTGTGAGCAGGACAGGGTGTCCCTCCCAAGGGGCT
GAGGTACCTTGGGCTCTTCTCTCCCTTAGATGCACGGCAACAAGCAGCAC
CTGCAGAAGGACTTCTTCCTGTACAATGCCTCCAAGGCTAGGAGCAGCCC
CTACATCAACATGCGGGAGGTGTCTGAGCGCTTCCGCCTGCTACACCAAT
ATTAA
(SEQ ID NO:2)
>CAPN3X3|Bta|Contig1 good se|17-678 snp 1 zebu
opp hom g266c
TTTTTGGGAAAAACATCAAACTCAATGTTCTCGAGGTAAAGCCTAACAGT
ACACTCCCCCCGCACTTTAAAATTTGAGGTGGAGGGATGGGATTGGATGG
GATGGGAAAGGAACATAAAGGGAAGCTCAGAGCTCAGCAGGATGTGGTCA
TGGGGAACGCATAGTGGGGGAAGGCTCTAAGGAAGCCTCAGTTATGGCTT
TATGCTGGAAAATCTGTACTTTCTAGGGGAGATCTGTGGGAAGGATGCTT
CTCAGGAAATCTTCC[c/g]TGTCTGGCATGCTGGGACTGGCCTTTTCTT
CTACGTTTTAGATATGTAGGGCTTGGCTCTTTTGTCTCATTGTGCATTCT
GATCTTGGGACTTCCTCTTGCAGTGGCTGCAGCTCACCATGTATGCCTGA
ACCAAGCTGGCCACATCGAAGGCATGGAGGATCACTCAGGATTTCAATTT
CACCCAACAGAGCTAGTCACTTACCTCAAAGGACACAGTTGCTGCACCCC
CAGGAAGCCTCCAGGCACCTCATCAGTCTAGTTCCTCCTCTACTCACTTC
CCTCCCAGCCTTAGGGCATCTGTCCATTTGTCAGGCCCAGCCTGACCCTT
CAGTTAAGGAATGGGGAAGGGAGAGCTGTGTTGTCCCTGTGCCACATGGA
GCCAAGTGCCTCTGTCTGCTTCCGCTAGCCACGACTACATTTA
(SEQ ID NO:3)

TABLE 9
The Taqman Probes and Primers for CAPN3

| | |
|---|---|
| CAPN3E6 (exon6) | |
| CAPN3E6100T | 6FAM-TGAGCCCATGTCC |
| | (SEQ ID NO:20) |
| CAPN3E6100G | VIC-TGAGCCCAGGTCC |
| | (SEQ ID NO:21) |
| CAPN3E6G100TU1 | ATGCCGACCGTCATTAGC |
| | (SEQ ID NO:22) |
| CAPN3E6G100TD1 | GAGTAGATGCCACTTGGGTTTC |
| | (SEQ ID NO:23) |

CAPN3JK (10^{th}/11^{th} introns)

| | |
|---|---|
| CAPN3JK-V2-642G | VIC-TACACGCTCACATGC |
| | (SEQ ID NO:24) |
| CAPN3JK-V2-642T | 6FAM-ACACTCTCACATGCT |
| | (SEQ ID NO:25) |
| CAPN3JKG642TU2 | ATTGCATGGCCTCCTGAC |
| | (SEQ ID NO:26) |
| CAPN3JKG642TD2 | CTCCAGAACACCTCTGGACTG |
| | (SEQ ID NO:27) |

CAPN3X3 (including the 3′UTR)

| | |
|---|---|
| CAPN3X3-266G | 6FAM-CTTCCGTGTCTGGC |
| | (SEQ ID NO:16) |
| CAPN3X3-266C | VIC-TCTTCCCTGTCTGGC |
| | (SEQ ID NO:17) |
| CAPN3X3G266CU2 | AGCCTCAGTTATGGCTTTATGC |
| | (SEQ ID NO:18) |
| 1-CAPN3X3G266CD1 | AGATCAGAATGCACAATGAGACA |
| | (SEQ ID NO: 19) |

TABLE 10
The effect of allele substitution of alleles at SNP at the CAPN3
gene on meat tenderness in zebu and zebu-cross breeds.

| | | | | | | | | | | | | | |
|---|---|---|---|---|---|---|---|---|---|---|---|---|---|
| Locus | mean0 | SE | mean1 | SE | mean2 | SE | N | Freq | a | k | a | tmax | PermP |

All Breeds

| | | | | | | | | | | | | | |
|---|---|---|---|---|---|---|---|---|---|---|---|---|---|
| capn3e6 | 0.01 | 0.07 | −0.03 | 0.03 | 0.02 | 0.02 | 2082 | 0.21 | −0.00 | 11.99 | 0.02 | 1.38 | 0.1671 |
| capn3jk | 0.01 | 0.02 | −0.02 | 0.03 | 0.13 | 0.07 | 1971 | 0.76 | −0.06 | 1.53 | −0.11 | 2.13 | 0.0379 |
| capn3x3 | 0.00 | 0.07 | −0.02 | 0.03 | 0.02 | 0.02 | 1768 | 0.19 | −0.01 | 2.98 | 0.01 | 1.11 | 0.2751 |
| cast3 | 0.14 | 0.06 | 0.08 | 0.03 | −0.08 | 0.03 | 1713 | 0.32 | 0.11 | 0.45 | 0.09 | 3.77 | 0.0001 |

Breed Specific

| | | | | | | | | | | | | | |
|---|---|---|---|---|---|---|---|---|---|---|---|---|---|
| capn3e6-BB | -0.02 | 0.10 | −0.11 | 0.05 | 0.08 | 0.05 | 614 | 0.29 | −0.05 | 3.03 | 0.01 | 2.61 | 0.0040 |
| capn3e6-BR | 0.06 | 0.12 | 0.10 | 0.05 | −0.02 | 0.03 | 838 | 0.15 | 0.04 | 2.05 | −0.02 | 1.86 | 0.0646 |
| capn3e6-SG | 0.05 | 0.14 | −0.06 | 0.05 | 0.04 | 0.04 | 630 | 0.20 | 0.01 | −18.96 | 0.07 | 1.52 | 0.1283 |
| capn3jk-BB | -0.10 | 0.06 | −0.02 | 0.05 | 0.19 | 0.08 | 566 | 0.52 | −0.15 | 0.45 | −0.15 | 2.96 | 0.0026 |
| capn3jk-BR | 0.02 | 0.03 | −0.05 | 0.07 | 0.79 | 0.82 | 789 | 0.92 | −0.38 | 1.19 | −0.76 | 1.81 | 0.0688 |
| capn3jk-SG | 0.04 | 0.04 | −0.00 | 0.05 | −0.18 | 0.08 | 616 | 0.78 | 0.11 | 0.67 | 0.15 | 1.59 | 0.1496 |
| capn3x3-BB | -0.06 | 0.10 | −0.10 | 0.05 | 0.08 | 0.05 | 562 | 0.29 | −0.07 | 1.72 | −0.02 | 2.40 | 0.0167 |
| capn3x3-BR | 0.15 | 0.15 | 0.11 | 0.06 | −0.02 | 0.03 | 660 | 0.15 | 0.08 | 0.53 | 0.05 | 1.84 | 0.0653 |
| capn3x3-SG | 0.03 | 0.13 | −0.05 | 0.06 | 0.04 | 0.04 | 546 | 0.16 | −0.00 | 16.23 | 0.05 | 1.07 | 0.3039 |
| cast3-BB | 0.11 | 0.08 | −0.00 | 0.05 | −0.12 | 0.06 | 556 | 0.44 | 0.11 | −0.01 | 0.11 | 2.30 | 0.0207 |
| cast3-BR | 0.18 | 0.16 | 0.16 | 0.05 | −0.06 | 0.04 | 646 | 0.23 | 0.12 | 0.78 | 0.07 | 3.42 | 0.0001 |
| casts-SG | 0.17 | 0.11 | 0.10 | 0.06 | −0.07 | 0.05 | 511 | 0.30 | 0.12 | 0.41 | 0.10 | 2.32 | 0.0212 |

Mean0 is the mean of the residual LDPF for genotype 0, SE is the standard error of the mean, mean1 is the mean for genotype 1, and so on. N is overall sample size, Freq is the allele frequency of allele 0, a is the additive effect of allele substitution, k is the dominance deviation, a is the average effect of allele substitution, tmax is the value of the t test between the most different genotypes and PermP is the P value determined by 100,000 permutations of the data.

### EXAMPLE 3

We sequenced the coding sequence of the CAPN3 gene in a range of animals of divergent breeds from mRNA of taurine and zebu cattle using ABI Big-Dye terminator chemistry on a 377 ABI DNA sequencer following the manufacturer's instructions (ABI, Foster City, Calif.). The consensus sequence is contained in Table 11. The CAPN3E6 SNP is at bp260.

TABLE 11

Additional single nucleotide polymorphisms in
the CAPN3 coding sequence.

>CAPN3|Bta| contig2 good seq bp 14-3051, start
bp 209, 1st stop bp 2675, coding SNP opp hom bp
260 ccca[gt]gtcc R -> M, bp 510 2 het agag[ag]cctc,
bp 759 opp hom caac[ct]taca, bp 2089 4 het
agac[ag]acac N -> D, bp 2235 1 het agaa[ct]gtcc,
bp 2289 opp horn tcac[ag]ctgg, bp 2313 opp hom
tgat[ct]gctc, bp 2330 3 het acag[at]tggc D -> V,
bp 2340 3 het ctgg[ag]agac, bp 2547 1 good het
actt[ct]gaca
TCCAGGAAAAAATGATGGCTTCCGGACCACAGCTCAGTTTTTAAGCTGGA
CGACCTTCTGATGGGTTTTAAACTTCGAATTGGATGTGGACATTTTTCTT
TCTGATGACAGAATCACTGGAACTTCCCCTTTGCAGTTGCTTCCTTTCCT
TAAAGGTAGCTGAATCTTGCTGTCTTTAAAAACCTTTTCTTTCCAAATTT
GCCTGCCATGCCGACCGTCATTAGCGCCTCTGTGGCCCCACGGACAGGGG
CTGAGCCCAtGTCCCCAGGGCCCATCGCCCAGGCAGCCCAGGACAAGGGC
ACCGAGGCAGGGGGTGGAAACCCAAGTGGCATCTACTCAGCCATCATCAG
CCGCAATTTTCCCATTATTGGGGTGAAAGAGAAGACATTCGAGCAGCTTC
ACAAGAAATGTCTGGAAAAGAAGGTTCTTTTTGTGGATCCTGAGTTCCCA
CCGGACGAGACCTCCCTGTTTTACAGCCAGAAGTTCCCCATCCAGTTCGT
CTGGAAGAGaCCTCCGGAAATTTGTGAGAATCCCCGATTTATCGTTGGTG
GAGCCAATAGAACTGACATCTGCCAAGGAGATCTAGGGGACTGCTGGTTT
CTTGCAGCCATCGCTTGCCTGACCTTGAACAAGCGTCTGCTTTTCCGGGT
CATACCCCATGATCAGAGTTTCACCGAAAACTACGCGGGGATTTTTCACT
TCCAGTTCTGGCGCTATGGAGACTGGGTGGACGTGGTTATTGATGACTGC
CTGCCAACCTACAACAATCAACTGGTTTTCACCAAATCCAACCATCGCAA
TGAGTTCTGGAGTGCTCTGCTGGAGAAGGCTTATGCTAAGCTCCATGGTT
CGTATGAAGCCCTGAAAGGTGGGAACACTACAGAGGCCATGGAGGACTTC
ACGGGAGGAGTGACAGAGTTTTTTGAAATCAAGGATGCTCCCAGAGACAT
GTACAAGATCATGAAGAAAGCCATCGAGAGGGGTTCCCTCATGGGCTGCT
CCATTGATGATGGCACAAACATGACCTATGGAACCTCTCCTTCTGGGCTG
AAAATGGGGGAGTTGATTGAGCGGATGGTGAGGAATATGGATAACTCGCG
GCTCAGGGACTCAGACCTCATCCCTGAGGGATGCTCAGATGACAGACCAA
CTCGGATGATTGTTCCAGTTCAGTTTGAGACAAGAATGGCCTGTGGGCTG
GTCAAAGGCCATGCCTACTCAGTCACTGGGCTGGAGGAGGCCCTGTACAA
GGGTGAGAAAGTGAAGCTGGTGCGGCTGCGGAACCCCTGGGGCCAGGTGG
AGTGGAATGGCTCCTGGAGTGACAGCTGGAAGGACTGGAGCTATGTGGAC
AAGGACGAGAAGGCCCGTTTGCAGCACCAGGTCACTGAGGATGGAGAGTT
CTGGATGTCCTACGATGATTTTATCTACCATTTCACAAAGCTGGAGATCT
GCAACCTCACAGCTGATGCCCTGGAGTCCGACAAGCTTCAGACTTGGACA
GTGTCCGTGAATGAGGGCCGCTGGGTGAGGGGCTGCTCTGCCGGAGGCTG
CCGCAACTTCCCAGACACTTTCTGGACCAACCCACAGTACCGTCTGAAGC
TCCTAGAGGAGGACGACGACCCCGATGATTCCGAGGTGATCTGTAGTTTC
CTGGTGGCTCTGATGCAGAAGAACCGGAGGAAGGACCGGAAGCTGGGGGC
TAACCTCTTCACCATCGGTTTCGCCATCTACGAGGTTCCCAAAGAGATGC
ACGGCAACAAGCAGCACCTGCAGAAGGACTTCTTCCTGTACAATGCCTCC
AAGGCTAGGAGCAGAACCTACATCAACATGCGGGAGGTGTCTGAGCGCTT
CCGCCTGCCTCCCAGCGAGTACGTCATTGTGCCCTCCACTTACGAGCCCC
ACCAGGAGGGCGAGTTCATCCTCCGGGTCTTCTCGGAAAAGAGGAACCTC
TCTGAGGAAGTTGAGAATACAATCTCTGTGGATCGGCCAGTGAAAAAGAA
AAAAAACAAGCCCATCATCTTTGTTTCAGACCGAGCAAACAGCAACAAGG
AGCTGGGTGTGGACCAGGAAACAGAGGAGGGAAAAGACaACACAAGCCCT
GATAAGCAAGCAAAATCCCCACAGCTAGAGCCTGGCAACACCGACCAGGA
AAGTGAGGAACAGCGGCAATTCCGGAATATTTTCAGGCAGATAGCAGGCG
ATGACATGGAGATCTGCGCAGATGAGCTCAAGAACGTCCTTAACAGAGTT
GTGAACAAACATAAGGACCTGAAGACACAAGGCTTCACgCTGGAGTCCTG
CCGTAGCATGATtGCTCTCATGGACACAGaTGGCTCTGGGaGACTGAACC
TGCAAGAGTTTCATCACCTCTGGAAGAAGATTAAGACGTGGCAGAAAATT
TTCAAACACTATGACACAGACCAATCTGGCACCATCAACAGCTACGAGAT
GCGCAATGCAGTCAAAGATGCAGGCTTCCACCTCAACAACCAGCTCTACG
ATATCATTACCATGCGCTATGCAGACAAGTACATGAATATTGACTTCGAC
AGTTTCATCTGCTGCTTTGTCAGGCTGGAGGGCATGTTCAGAGCTTTCAA
TGCATTTGACAAGGATGGGGACGGTATCATCAAACTCAATGTTCTCGAGT
GGCTGCAGCTCACCATGTATGCCTGAACCAAGCTGGCCACATCGAAGGCA
TGGAGGATCACTCAGGATTTCAATTTCACCCAACAGAGCTAGTCACTTAC
CTCAAAGGACACAGTTGCTGCACCCCCAGGAAGCCTCCAGGCACCTCATC
AGTCTAGTTCCTCCTCTACTCACTTCCCTCCCAGCCTTAGGGCATCTGTC
CATTTGTCAGGCCCAGCCTGACCCTTCAGTTAAGGAATGGGGAAGGGAGA
GCTGTGTTGTCCCTGTGCCACATGGAGCCAAGTGCCTCTGTCTGCTTCCG
CTAGCCACAGGCCAGTGAGAGCTTTGCTTGGAGTCTGATGGCCTCAGCTC
TGAAGACAAGTACTCTCCTTAGATGCTTGCAGCTGTTTGCAGAAGCATTT
GCCAATGGCAAAAA
(SEQ ID NO:4)

Each polymorphism shown in SEQ ID NO: 4 is set out singly in position order from 5′ to 3′ with flanking sequence in SEQ ID NOs: 5 to 13 except the bp260 polymorphism which is set out in SEQ ID NO: 1 and identified above as the CAPN3E6 polymorphism.

TABLE 12
The lab names of the SNP used in examples, such as CAPN3E6,
CAPN3JK and CAPN3X3 have standardised names (den Dunnen and
Antonarakis 2000), as do the SNP in SEQ ID NO: 4. These are
as follows in order from 5′ to 3′ in the gene:

| | | Coding sequence |
|---|---|---|
| Lab Name | Standard Name | effect |
| CAPN3E6 | CAPN3: c.53T > G | Met18Arg |
| CAPN3-510 | CAPN3: c.303A > G | Arg no change |
| CAPN3-759 | CAPN3: c.552C > T | Thr no change |
| CAPN3JK | CAPN3: c.1538 + 225G > T | Intronic |
| CAPN3-2089 | CAPN3: c.1882A > G | Asn628Asp |
| CAPN3-2235 | CAPN3: c.2028C > G | Asn no change |
| CAPN3-2289 | CAPN3: C.2082G > A | Thr no change |
| CAPN3-2313 | CAPN3: c.2106T > C | Ile no change |
| CAPN3-2330 | CAPN3: c.2123A > T | Val708Asp |
| CAPN3-2340 | CAPN3: c.2133A > G | Gly no change |
| CAPN3-2547 | CAPN3: c.2339C > T | Phe no change |
| CAPN3X3 | CAPN3: c.2443 − 103G > C | Intronic |

To determine which of the new SNP derived from sequencing the CAPN3 gene are associated with meat tenderness, Taqman Assays were constructed using the Taqman methodology. The same samples were used as in Example 1, to determine which alleles had more favourable tenderness. In each case (Table 13), the rarer allele always had a less tender or more tough average phenotype, even if these were not statistically significant. In Table 13 the rarer alleles are CAPN3-759 T, CAPN3-2289 A, and CAPN3-2313 C.

TABLE 13
Associations between meat tenderness and CAPN3 SNP.

| Locus | x0 | SE | x1 | SE | x2 | SE | N | Freq0 | alpha | tmax | PermP |
|---|---|---|---|---|---|---|---|---|---|---|---|
| capn3-759 | 0.34 | 0.18 | 0.93 | 0.42 | 1.54 | 2.56 | 86 | 0.89 | −0.61 | 1.36 | 0.1700 |
| capn3-2289 | 0.69 | 1.70 | 1.44 | 0.38 | 0.52 | 0.12 | 233 | 0.06 | −0.65 | 2.43 | 0.0117 |
| capn3-2313 | 0.97 | 0.57 | 0.80 | 0.27 | 0.52 | 0.13 | 236 | 0.15 | 0.19 | 0.97 | 0.3488 |

Alpha, the average effect of allele substitution is measured in kg of peak force.

Similarly, the CAPN3:c.1882G allele, which is the rarer allele at the CAPN3:c.1882A>G SNP, and the CAPN3:c.2123T allele, which is the rarer allele at the CAPN3:c.2123A>T SNP, will cause an increase in meat toughness.

Furthermore, the CAPN3:c.303G allele, which is the rarer allele at the CAPN3:c.303A>G SNP, the CAPN3:c.2028G allele, which is the rarer allele at the CAPN3:c.2028C>G SNP, and the CAPN3:c.2339T allele, which is the rarer allele at the CAPN3:c.2339C>T SNP, will cause an increase in meat toughness.

### REFERENCES

The contents of the following documents are incorporated herein through reference:
Altschul, S. F., Gish, W., Miller, W., Myers, E. W. & Lipman, D. J. Basic local alignment search tool. J. Mol. Biol. 215, 403-410 (1990).
Barendse, W. 2001. DNA markers for meat tenderness. Patent WO02064820 (Patent Application PCT/AU02/00122).
Barendse, W. The transition from quantitative trait loci to diagnostic test in cattle and other livestock. Aust. J. Exp. Agr. 45, 831-836 (2005).
Barendse, W., 1997. Assessing lipid metabolism. Patent application PCT/AU98/00882
Barendse, W. and Fries, R., 1999. Genetic linkage mapping, the gene maps of cattle, and the lists of loci, pp. 329-364 in The genetics of cattle, edited by R. Fries and A. Ruvinsky, CABI Publishing, Wallingford.
Barendse, W. and Harrison, B., (2001) The analysis of effects on instron compression and adhesion in the semitendinosus muscle of genotypes at the candidate gene Lysyl Oxidase (LOX) in cattle of diverse breeds. CRC commercial-in-confidence report.
Barendse, W., Harrison, B. and Li, Y. 2000. The analysis of effects on peak-force of genotypes at the candidate gene Calpastatin in cattle of diverse breeds. Confidential Report of the Beef Quality CRC.
Barendse, W., Bunch, R. J., Harrison, B. E. & Thomas, M. B. The growth hormone GH1:c.457C>G mutation is associated with relative fat distribution in intra-muscular and rump fat in a large sample of Australian feedlot cattle. Anim. Genet. 37, 211-214 (2006).
Carlborg, O., Jacobsson, L., Ahgren, P., Siegel, P. & Andersson, L. Epistasis and the release of genetic variation during long-term selection. Nat. Genet. 38, 418-420 (2006).
Casas, E. et al. Quantative trait loci affecting growth and carcass composition of cattle segregating alternate forms of myostatin. J. Anim. Sci. 78, 560-569 (2000).
Chen, X. N., B. Zehnbauer, A. Gnirke, and P. Y. Kwok. 1997. Fluorescence energy transfer detection as a homogeneous DNA diagnostic method. Proc. Natl. Acad. Sci. (USA) 94: 10756-10761.
Chung, H. Y., Davis, M. E. and Hines, H. C. 1999. A DNA polymorphism of the bovine calpastatin gene detected by SSCP analysis. Animal Genetics 30, 80.
Chung, H. Y., Davis, M. E., Hines, H. C. and Wulf, D. M. 1999. Relationship of a PCR-SSSCP at the bovine Calpastatin locus with Calpastatin activity and Meat Tenderness. Ohioline Bulletin Special Circular 170-99. http://www.ag.ohio-state.edu/˜ohioline/sc170/sc170_{—}3.html
Coleman, J. B., Cucca, F., Hearne, C. M., Cornall, R. J., Reed, P. W., Ronningen, K. S., Undlien, D. E., Nistico, L., Buzzetti, R., Tosi, R., Pociot, F., Nerup, J., ornelis, F., Barnett, A. H., Bain, .C., and Todd, J. A. 1995. Linkage disequilibrium mapping of a type 1 diabetes susceptibility gene (IDDM7) to chromosome 2q31-q33. Nature Genetics 9, 80-85.
Cronlund, A. L., Smith, B. D., Kagan, H. M. 1985. Binding of lysyl oxidase to fibrils of type I Collagen. Connective Tissue Research 14, 109-119.
den Dunnen, J. T. and S. E. Antonarakis. 2000. Mutation nomenclature extensions and suggestions to describe complex mutations: a discussion. Hum. Mut. 15: 7-12.
Drinkwater, R. D., Harrison, B., Byrne, K., Botero, F. A., Knight, M., Davis, G. P., Lenane, I., Li, Y., Kuipers, R., and Moore, S. S. 1999. Candidate genes for Meat Quality, draft report for the 1998-1999 research program. Cattle and Beef CRC Commercial-In-Confidence Report.
Drinkwater, R. D. et al. Detecting quantitative trait loci affecting beef tenderness on bovine chromosome 7 near calpastatin and lysyl oxidase. Aust. J. Exp. Agr. 46, 159-164 (2006).
Ekholm, E. C., Ravanti, L., Kahari, V., Paavolainen, P. and Penttinen, R. P. 2000. Expression of extracellular matrix genes: transforming growth factor (TGF)-beta1 and ras in tibial fracture healing of lathyritic rats. Bone 27, 551-557.
Ewing, B., Hillier, L., Wendl, M. C. & Green, P. Base-calling of automated sequencer traces using phred. I. Accuracy assessment. Genome Res. 8, 175-185 (1998).
Farnir, F., W. Coppieters, J. J. Arranz, P. Berzi, N. Cambisano, B. Grisart, L. Karim, F. Marcq, L. Moreau, M. Mni, C. Nezer, P. Simon, P. Vanmanshoven, D. Wagenaar, and M. Georges. 2000. Extensive genome-wide linkage disequilibrium in cattle. Genome Res. 10: 220-227.
Fisher, R. A. The correlation between relatives on the supposition of Mendelian inheritance. Trans. Roy. Soc. Edin. 52, 399-433 (1918).
Garcia, M. D. et al. Significant association of the calpastatin gene with fertility and longevity in dairy cattle. Anim. Genet. 37, 304-305 (2006).
Geesink, G. H. and Koohmaraie, M. 1999. Postmortem proteolysis and calpain/calpastatin activity in callipyge and normal lamb biceps femoris during extended postmortem storage. J. Anim. Sci. 77, 1490-1501.
Giampuzzi, M., Botti, G., Cilli, M., Gusmano, R., Borel, A., Sommer, P., Di Donato, A. 2001. Down regulation of lysyl oxidase induced tumorigenic transformation in NRK-49F cells characterized by constitutive activation of Ras proto-oncogene. Journal of Biological Chemistry Eepub ahead of print].
Gilmour, A. R., Thompson, R. & Cullis, B. R. Average information REML: An efficient algorithm for variance parameter estimation in linear mixed models. Biometrics 51, 1440-1450 (1995).
Goll, D. E., Thompson, V. F., Li, H. Q., Wei, W. & Cong, J. Y. The calpain system. Physiological Reviews 83, 731-801 (2003).
Gordon, D., Abajian, C. & Green, P. Consed: a graphical tool for sequence finishing. Genome Res. 8, 195-202 (1998).
Hardenbol, P., J. Baner, M. Jain, M. Nilsson, E. A. Namsaraev, G. A. Karlin-Neumann, H. Fakhrai-Rad, M. Ronaghi, T. D. Willis, U. Landegren, and R. W. Davis. 2003. Multiplexed genotyping with sequence-tagged molecular inversion probes. Nature Biotech. 21: 673-678.
Hardenbol, P., F. L. Yu, J. Belmont, J. MacKenzie, C. Bruckner, T. Brundage, A. Boudreau, S. Chow, J. Eberle, A. Erbilgin, M. Falkowski, R. Fitzgerald, S. Ghose, O. Iartchouk, M. Jain, G. Karlin-Neumann, X. H. Lu, X. Miao, B. Moore, M. Moorhead, E. Namsaraev, S. Pasternak, E. Prakash, K. Tran, Z. Y. Wang, H. B. Jones, R. W. Davis, T. D. Willis, and R. A. Gibbs. 2005. Highly multiplexed molecular inversion probe genotyping: over 10,000 targeted SNPs genotyped in a single tube assay. Genome Res. 15: 269-275.
Hetzel., D. J. S and Davis, G. P. 1999. Gene markers for carcass, meat quality and live animal traits. Final report CSIRO Tropical Agriculture and CRC for Meat Quality. Report finalised by Y. Li.
Ilian, M. A., Bekhit, A. E-D., Bickerstaffe, R. 2004. The relationship between meat tenderization, myofibril fragmentation and autolysis of calpain 3 during post-mortem aging. Meat Science 66, 387-397.
Ilian, M. A., Morton, J. D., Bekhit, A. E.-D., Roberts, N., Palmer, B., Sorimachi, H. and Bickerstaffe, R. Journal of Agricultural and Food Chemistry 49, 1990-1998.
James, T., Matzelle, D., Bartus, R., Hogan, E. L. & Banik, N. L. New inhibitors of calpain prevent degradation of cytoskeletal and myelin proteins in spinal cord in vitro. J. Neurosci. Res. 51, 218-222 (1998).
J. Henshall and M. Goddard (1999) Multiple-trait mapping of quantitative trait loci after selective genotyping using logistic regression. Genetics 151:885-894.
Livak, K. J. 2003. SNP genotyping by the 5′-nuclease reaction. In Meth. Mol. Biol. (ed. P.-Y. Kwok), pp. 129-147. Humana Press, Totowa N.J.
Y. Li (2000) CRC Molecular Genetics Program Annual Report. CRC commercial-in-confidence report.
Lonergan, S. M., Ernst, C. W., Bishop, M. D., Calkins, C. R., and Koohmaraie, M. 1995. Relationship of restriction fragment length polymorphisms (RFLP) at the bovine calpastatin locus to calpastatin activity and meat tenderness. J. Anim. Sci 73, 3608-3612.
Luo, L. J. et al. Overdominant epistatic loci are the primary genetic basis of inbreeding depression and heterosis in rice. II. Grain yield components. Genetics 158, 1755-1771 (2001).
Lynch, M. & Walsh, J. B. Genetics and Analysis of Quantitative Traits (Sinauer Associates, Inc., Sunderland, Mass., 1998).
Koohmaraie, M. Biochemical factors regulating the toughening and tenderization processes of meat. Meat Sci. 43, 193-201 (1996).
Koohmaraie, M. 1994. Muscle proteinases and meat aging meat Sci. 36:93.
McRae, A. F., J. C. McEwan, K. G. Dodds, T. Wilson, A. M. Crawford, and J. Slate. 2002. Linkage disequilibrium in domestic sheep. Genetics 160: 1113-1122.
Nellaiappan, K., Risitano, A., Liu, G., Nicklas, G. and Kagan, K. M. 2000. Fully processed lysyl oxidase catalyst translocates from the extracellular space into nuclei of aortic smooth-muscle cells. Journal of Cell Biochemistry 79, 576-582.
Nickerson, D. A., To be, V. O. & Taylor, S. L. PolyPhred: Automating the detection and genotyping of single nucleotide substitutions using fluorescence-based resequencing. Nucl. Acids Res. 25, 2745-2751 (1997).
Normeman, D. and M. Koohmaraie. 1999. Molecular cloning and mapping of the bovine and ovine skeletal muscle-specific calpains. Anim. Genet. 30: 456-458.
Nsengimana, J., P. Baret, C. S. Haley, and P. M. Visscher. 2004. Linkage disequilibrium in the domesticated pig. Genetics 166: 1395-1404.
Normeman, D., Kappes, S. M. and Koohmaraie, M. 1999. Rapid communication: a polymorphic microsatellite in the promotor region of the bovine calpastatin gene. J. Anim. Sci 77, 3114-3115.
Olson, T. A. in The genetics of cattle (eds. Fries, R. & Ruvinsky, A.) 33-53 (CAB International, Wallingford, 1999).
Page, B. T. et al. Evaluation of single-nucleotide polymorphisms in CAPN1 for association with meat tenderness in cattle. J. Anim. Sci. 80, 3077-3085 (2002).
Perry, D., Shorthose, W. R., Ferguson, D. M. & Thompson, J. M. Methods used in the CRC program for the determination of carcass yield and beef quality. Aust. J. Exp. Agr. 41, 953-957 (2001).
Robinson, D. L., Ferguson, D. M., Oddy, V. H., Perry, D. & Thompson, J. Genetic and environmental influences on beef tenderness. Aust. J. Exp. Agr. 41, 997-1003 (2001).
Slee, R. B., Hillier, S. G., Largue, P., Harlow, C. R., Miele, G. and Clinton, M. 2001. Differentiation-dependent expression of connective tissue growth factor and lysyl oxidase messenger ribonucleic acids in rat granulosa cells. Endocrinology 142, 1082-1089.
Storm, N., B. Darnhofer-Demar, D. van den Boom, and C. P. Rodi. 2002. MALDI-TOF mass spectrometry-based SNP genotyping. Meth. Mol. Biol. 212: 214-262.
Tenesa, A., S. A. Knott, D. Ward, D. Smith, J. L. Williams, and P. M. Visscher. 2003. Estimation of linkage disequilibrium in a sample of the United Kingdom dairy cattle population using unphased genotypes. J. Anim. Sci. 81: 617-623.
Terwilliger, J. D. 1995. A powerful likelihood method for the analysis of linkage disequilibrium between trait loci and one or more polymorphic marker loci. American Journal of Human Genetics 56, 777-787.
Upton, W., Burrow, H. M., Dundon, A., Robinson, D. L. & Farrell, E. B. CRC breeding program design, measurements and database: methods that underpin CRC research results. Aust. J. Exp. Agr. 41, 943-952 (2001).
Wang, D. L., Zhu, J., Li, Z. K. & Paterson, A. H. Mapping QTLs with epistatic effects and QTLx environment interactions by mixed linear model approaches. Theor. Appl. Genet. 99, 1255-1264 (1999).
Whipple, G., Koohmaraie, M., Dikeman, M. E., Crouse, J. D., Hunt, M. C., Klemm, R. D. 1990. Evaluation of attributes that affect longissimus muscle tenderness in Bos taurus and Bos indicus cattle. J.Anim. Sci. 68, 2716-2728.
White, S. N. et al. A new single nucleotide polymorphisms in CAPN1 extends the current tenderness marker test to include cattle of Bos indicus, Bos taurus, and crossbred descent. J. Anim. Sci. 83, 2001-2008 (2005).
Woodward, B. W., DeNise, S. K., and Marchello, J. A. 2000. Evaluation of calpastatin activity measures in ante-and postmortem muscle from half-sib bulls and steers. J.Anim. Sci. 78, 804-809.

1. A method for assessing the tenderness of meat from an animal, comprising the step of testing the animal for a genetic marker in the calpain3 (CAPN3) gene associated with Warner-Bratzler peak force variation or for a genetic marker located other than in CAPN3 which shows allelic association therewith.

2. A method as claimed in claim 1 wherein the genetic marker is located in an intron.

3. A method as claimed in claim w 2 wherein the genetic marker is located in intron 10 or 11.

4. A method as claimed in claim 3 wherein the genetic marker is CAPN3:C.552C>T.

5. A method as claimed in claim 1 wherein the genetic marker is located in the 3′UTR of CAPN3.

6. A method as claimed in claim 5 wherein the genetic marker is CAPN3:C.2443-103G>C.

7. A method as claimed in claim 1 wherein genetic marker is located in the coding sequence of the CAPN3 gene.

8. A method as claimed in claim 7 wherein the genetic marker is selected from the group consisting of CAPN3:c.303A>G, CAPN3:c.552C>T, CAPN3:c.1882A>G, CAPN3:c.2028C>G, CAPN3:c.2082G>A, CAPN3:c.2106T>C, CAPN3:c.2123A>T, CAPN3:c.2133A>G and CAPN3:c.2339C>T.

9. A method as claimed in claim 1, further comprising the step of testing for one or more additional genetic markers associated with Warner-Bratzler peak force variation.

10. A method as claimed in claim 9 further comprising the step of testing for any combination of two or more of CAPN3:C.552C>T, CAPN3:C.2443-103G>C,CAPN3:c.303A>G, CAPN3:c.552C>T, CAPN3:c.1882A>G, CAPN3:c.2028C>G, CAPN3:c.2082G>A, CAPN3:c.2106T>C, CAPN3:c.2123A>T, CAPN3:c.2133A>G and CAPN3:c.2339C>T.

11. A genetic marker for meat tenderness in an animal which is a polymorphism in the calpain3 gene.

12. A genetic marker as claimed in claim 11 which is CAPN3:C.552C>T.

13. A genetic marker as claimed in claim 11 which is CAPN3:C.2443-103G>C.

14. A genetic marker as claimed in claim 11 selected from the group consisting of CAPN3:c.303A>G, CAPN3:c.552C>T, CAPN3:c.1882A>G, CAPN3:c.2028C>G, CAPN3:c.2082G>A, CAPN3:c.2106T>C, CAPN3:c.2123A>T, CAPN3:c.2133A>G and CAPN3:c.2339C>T.

15. A method as claimed in claim 1 further comprising for selecting an animal likely to yield meat of improved tenderness by selecting animals in which said genetic marker is present.

16. A method as claimed in claim 15, further comprising using the selected animal for breeding.

17. An oligonucleotide probe for amplification of a genetic marker in the calpain3 (CAPN3) gene associated with Warner-Bratzler peak force variation or for a genetic marker located other than in CAPN3 which shows allelic association therewith.

18. A kit for use in a method as claimed in claim 1, comprising oligonucleotide probes for amplification of at least one genetic marker associated with meat tenderness in the calpain3 (CAPN3) gene associated with Warner-Bratzler peak force variation or for a genetic marker located other than in CAPN3 which shows allelic association therewith, and means for amplifying DNA.
